(19) United States
(12) Patent Application Publication
(10) Pub. No.: US 2002/0082421 A1
(43) Pub. Date: Jun. 27, 2002

(54) Novel solid salt forms of N-[2-[4-[2-(1- methylethoxy)phenyl]-1-piperazinyl]-2-oxo-1piperidineacetamide

(76) Inventors: Ahmed F. Abdel-Magid, Ambler, PA (US); Daniel J. Korey, Yardley, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/950,502

(22) Filed: Sep. 11, 2001

Related U.S. Application Data
(60) Provisional application No. 60/232,532, filed on Sep. 14, 2000.

(51) Int. Cl. C 07D 4 1/02

(52) U.S. Cl. 544/360

(57) ABSTRACT

The present invention relates to novel solid salt forms of N-[2-[4-[2-(1-methylethoxy)phenyl]-1-piperazinyl]ethyl]-2-oxo-1-piperidineacetamide and processes for their preparation.

### CROSS REFERENCE TO RELATED APPLICATION

This application claims priority from U.S. provisional application Serial No. 60/232,532, filed Sep. 14, 2000, which is hereby incorporated by reference.

### BACKGROUND OF THE INVENTION

### FIELD OF THE INVENTION

The present invention relates to novel solid salt forms of N-[2-[4-[2-(1-methylethoxy)phenyl]-1-piperanzinyl]ethyl]-2-oxo-1-piperidineacetamide and processes for their preparation. More particularly, the present invention is directed to solid hydrobromic, hydrochloric, hydroiodic, cyclohexanesulfamic and sulfuric acid salts.

U.S. Pat. No. 6,071,915 (Issued Jun. 6, 2000) discloses a class of novel aryl substituted piperazines and pharmaceutically acceptable salts thereof, compounds which selectively inhibit binding to the α-1a adrenergic receptor, a receptor which has been implicated in benign prostatic hyperplasia. One of these compounds is N-[2-[4-[2-(1-methylethoxy)phenyl]-1-piperazinyl]ethyl]-2-oxo-1-piperidineacetamide, a compound of formula (I).

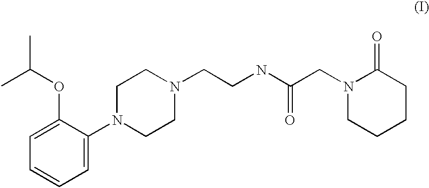

The compound of formula (I) as a free base is an oil. For use as a pharmaceutical agent, it is desirable to have the compound of formula (I) present as a solid salt. Solid salts are preferred as they are generally more soluble in water, are generally more bioavailable and are easier to handle in the production of tablets and other dosage formulations.

### BRIEF SUMMARY OF THE INVENTION

The present invention relates to novel solid salt forms of the compound of formula (I), more particularly, solid hydrobromic, hydrochloric, hydroiodic, cyclohexanesulfamic and sulfuric acid salts.

In an embodiment of the present invention the solid salts are mono-hydrobromide, mono-hydrochloride, mono-hydriodide, mono-cyclohexanesulamate and mono-sulfate salts of the compound of formula (I).

In an aspect of the present invention are crystalline forms of the mono-hydrobromide, mono-hydrochloride, mono-hydriodide, mono-cyclohexanesulamate and mono-sulfate salts of the compound of formula (I)

In another aspect of the present invention, the mono-hydrochloride salt may be present as an anhydrous or di-hydrate form, and the mono-sulfate salt may be present as a mono-hydrate form.

In yet another aspect of the present invention, the mono-hydrobromide salt may be present as unsolvated Form A, unsolvated Form C, solvated Form B or amorphous Form D.

In yet another aspect, the claimed invention relates to processes for the preparation of said novel solid salts of the compound of formula (I).

### DETAILED DESCRIPTION OF THE INVENTION

The compound of formula (I) as a free base may be prepared according to the process outlined in Scheme 1.

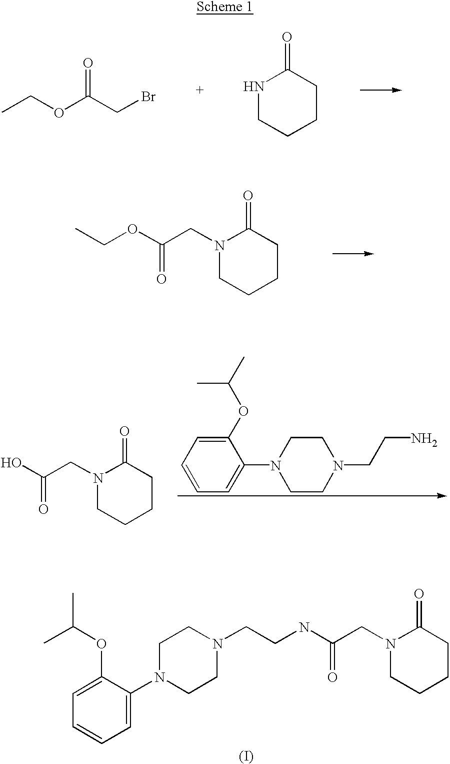

More particularly, bromoacetic acid ethyl ester (a known compound) is reacted with 2-piperidone (a known compound) in the presence of a strong non-aqueous base such as NaH, lithium hexamethyldisilazide, and the like, preferably NaH, at a temperature in the range of about 5-15° C., to yield 1-(ethoxycarbonylmethyl)-2-piperidone.

1-(ethoxycarbonylmethyl)-2-piperidone is treated with an aqueous alkali hydroxide such as NaOH, LiOH, and the like, preferably NaOH, to yield the corresponding acid, 2-oxo-1-piperidine acetic acid.

The 2-oxo-1-piperidine acetic acid is preferably not isolated and is reacted with 4-[2-(1-methylethoxy)phenyl]-1-piperazine ethanamine (a known compound), in the presence of a base such as NaOH, LiOH, sodium carbonate, and the like, preferably NaOH, in the presence of a coupling agent such as N,N′-dicyclohexylcarbodiimide (DCC), N,N′-diisopropylcarbodiimide (DIC), and the like, preferably DCC, to yield the compound of formula (I).

As used herein, the term “mono-sulfate salt of the compound of formula (I)” shall mean a sulfate salt of the compound of formula (I) wherein the molar ratio of the compound of formula (I) to the sulfate ion is 1:1.

As used herein, the abbreviation “KF” shall mean the weight percent of water in a product, as determined by the Karl-Fischer test.

As used herein, unless otherwise noted, the term “anti-solvent” shall refer to a solvent which does not dissolve a specific substance and is added to a solution of said substance, directly or by vapor diffusion, to cause precipitation of said substance.

The invention relates to novel solid salt forms of the compound of formula (I), more particularly, solid hydrobromic, hydrochloric, hydroiodic, cyclohexanesulfamic and sulfuric acid salts.

In an embodiment of the present invention the solid salt forms are mono-hydrobromide, mono-hydrochloride, mono-hydroiodide, mono-cyclohexanesulfamate and mono-sulfate salts of the compound of formula (I).

In an aspect of the present invention are crystalline forms of the mono-hydrobromide, mono-hydrochloride, mono-hydroiodide, mono-cyclohexanesulamate and mono-sulfate salts of the compound of formula (I)

In an aspect of the present invention, the mono-hydrochloride salt may be present as an anhydrous or di-hydrate form. In another aspect of the present invention, the mono-sulfate salt may be present as a mono-hydrate form.

In yet another aspect of the present invention, the mono-hydrobromide salt may be present as unsolvated Form A, unsolvated Form C, solvated Form B or amorphous Form D.

In yet a further aspect, the claimed invention relates to processes for the preparation of said novel solid salts of the compound of formula (I).

The solid hydrobromide salt of the compound of formula (I), as unsolvated Form A, may be prepared by reacting the compound of formula (I) with hydrogen bromide gas (HBr), wherein the HBr is present in an amount in the range of about 0.95-1.2 molar equivalents, preferably in the range of about 0.95 to 1.1 molar equivalents, in a polar organic solvent such as methanol, ethanol, ethyl acetate, isopropyl alcohol, and the like or mixtures thereof, preferably anhydrous ethanol or isopropyl alcohol.

The solid hydrobromide salt of the compound of formula (I), as amorphous Form D, may be prepared by dissolving the unsolvated Form A in an organic solvent such as dichloromethane, methanol, and the like, and rapidly evaporating the solvent, for example by rotary evaporator.

Alternatively, amorphous Form D may be prepared by melting Form A and quenching the melt, for example by pouring the melt onto a cold plate.

The solid hydrobromide salt of the compound of formula (I), as unsolvated Form C, may be prepared by heating amorphous Form D at a temperature in the range of about 95° C. to about 115° C., preferably at a temperature of about 100° C. for about 3 hours; and allowing the sample to cool at room temperature.

The solid hydrobromide salt of the compound of formula (I), as solvated Form B, may be prepared by dissolving Form A of the hydrobromide salt in an solvent such as acetonitrile, methanol, water, and the like, and slowly evaporating the solvent, for example, by allowing the solvent to evaporate under room temperature.

By contrast, Form A of the hydrobromide salt is recovered when Form A of the hydrobromide salt is dissolved in a solvent such as methanol, ethanol, N,N-dimethylformamide, dichloromethane, and the like, and the solid is precipitated by cooling, by use of an antisolvent or by vapor diffusion crystallization with an antisolvent. Suitable solvent:antisolvent pairs for recovering Form A by crystallization with an anti-solvent include methnaol:acetone, water:acetone, ethanol:ethyl acetate and methanol:ethyl acetate. Suitable solvent:antisolvent pairs for recovering Form A by vapor diffusion crystallization include dichloromethane:acetone, dichloromethane:diethyl ether, dichloromethane:hexanes, dichloromethane:tetrahydrofuran and N,N-dimethylformamide:toluene.

The solid hydrochloride salt of the compound of formula (I) may be prepared by reacting the compound of formula (I) with hydrogen chloride gas (HCl), preferably in an amount in the range of about 0.95 to 1.05 molar equivalents, in a polar organic solvent such as methanol, ethanol, ethyl acetate, isopropyl alcohol, and the like, or mixtures thereof, preferably in a mixture of ethyl acetate/methanol.

The solid hydroiodide salt of the compound of formula (I) may be prepared by reacting the compound of formula (I) with concentrated hydroiodic acid, preferably in an amount in the range of about 0.95 to 1.05 molar equivalents, in a polar organic solvent such as methanol, ethanol, ethyl acetate, isopropyl alcohol or mixtures thereof, preferably in a mixture off ethyl acetate/methanol.

The solid cyclohexanesulfamate salt of the compound of formula (I) may be prepared by reacting the compound of formula (I) with cyclohexanesulfamic acid, preferably in an amount in the range of about 0.95 to 1.05 molar equivalents, in a polar organic solvent such as methanol, ethanol, ethyl acetate, isopropyl alcohol or mixtures thereof, preferably in a mixture of ethyl acetate/methanol.

The solid sulfate salt of the compound of formula (I) may be prepared by reacting the compound of formula (I) with concentrated sulfuric acid, preferably in an amount in the range of about 0.95 to 1.05 molar equivalents, in a polar organic solvent such as methanol, ethanol, ethyl acetate, isopropyl alcohol or mixtures thereof, preferably in a mixture of ethyl acetate/methanol.

Preferably, the solid salts of the compound of formula (I) are recrystallized from a polar organic solvent such as methanol, ethanol, ethyl acetate, isopropyl alcohol, and the like, or mixtures thereof.

The crystalline salt forms of the compound of formula (I), N-[2-[4-[2-(1-methylethoxy)phenyl]-1-piperanzinyl]ethyl]-2-oxo-1-piperidineacetamide, may be characterized by their respective X-ray diffraction patterns.

Unless otherwise noted, X-ray diffraction patterns were measured on a Siemens D5000 powder x-ray diffractometer with the following settings:

CuKα radiation, 35 mA, 40 kV

Parallel beam optics with thin film attachment

Scan 0.02°2θ per second

The X-ray diffraction patterns listed in Table 2, Table 3 and Table 4 were measured on a Shimadzu XRD-600 X-ray powder diffractometer with the following settings:

CuKα radiation, 40 mA, 40 kV

Divergence and scattering slits set at 1°, receiving slit set at 0.15 mm

Scanning from 2.5-40° 2θ, at 3°/min (0.4 sec/0.02° step)

The crystalline hydrobromide salt of the compound of formula (I), unsolvated Form A, may be characterized by its X-ray diffraction pattern, comprising the peaks:

TABLE 1

| | | Relative Intensity |
|---|---|---|
| ANGLE °2θ | d-Spacing (Å) | (%) |
| 6.583 | 13.416 | 65.7 |
| 7.293 | 12.111 | 29.2 |
| 8.919 | 9.906 | 14 |
| 12.208 | 7.244 | 15 |
| 13.039 | 6.784 | 47.8 |
| 13.624 | 6.494 | 17.1 |
| 13.988 | 6.326 | 11.9 |
| 14.867 | 5.954 | 15.2 |
| 16.343 | 5.419 | 25.9 |
| 16.781 | 5.279 | 10.2 |
| 17.189 | 5.154 | 33.3 |
| 18.006 | 4.922 | 100 |
| 19.354 | 4.582 | 24.9 |
| 20.067 | 4.421 | 66.7 |
| 20.32 | 4.367 | 23.7 |
| 20.884 | 4.25 | 47.4 |
| 21.131 | 4.201 | 27.6 |
| 21.545 | 4.121 | 20.1 |
| 21.939 | 4.048 | 46.1 |
| 22.665 | 3.92 | 39.1 |
| 23.663 | 3.757 | 16.7 |
| 24.272 | 3.664 | 28.7 |
| 24.599 | 3.616 | 53.1 |
| 25.006 | 3.558 | 40.8 |
| 25.379 | 3.507 | 17.4 |
| 26.372 | 3.377 | 29.7 |
| 26.755 | 3.329 | 62.6 |
| 27.482 | 3.243 | 23 |
| 27.85 | 3.201 | 59 |
| 28.636 | 3.115 | 12.8 |
| 29.54 | 3.021 | 10.4 |
| 29.722 | 3.003 | 15.9 |
| 30.354 | 2.9422 | 21.7 |
| 31.18 | 2.8662 | 13.5 |
| 31.637 | 2.8257 | 13 |
| 31.956 | 2.7983 | 11.9 |
| 33.003 | 2.7119 | 11.3 |
| 33.334 | 2.6857 | 14.8 |
| 34.698 | 2.5832 | 10.6 |

The crystalline hydrobromide salt of the compound of formula (I), unsolvated Form A, may be characterized by its X-ray diffraction pattern, comprising the major peaks:

TABLE 2

| | | Relative Intensity |
|---|---|---|
| ANGLE °2θ | d-Spacing (Å) | (%) |
| 6.6 | 13.34 | 33 |
| 13.0 | 6.79 | 31 |
| 17.2 | 5.16 | 26 |
| 17.8 | 5.00 | 28 |
| 18.0 | 4.93 | 100 |
| 20.1 | 4.42 | 63 |
| 20.9 | 4.25 | 40 |
| 21.9 | 4.05 | 35 |
| 22.6 | 3.92 | 37 |
| 24.3 | 3.67 | 29 |
| 24.6 | 3.62 | 46 |
| 25.0 | 3.56 | 45 |
| 26.4 | 3.38 | 40 |
| 26.7 | 3.33 | 66 |
| 27.8 | 3.21 | 60 |

The crystalline hydrobromide salt of the compound of formula (I), solvated Form B, may be characterized by its X-ray diffraction pattern, comprising the peaks:

TABLE 3

| | | Relative Intensity |
|---|---|---|
| ANGLE °2θ | d-Spacing (Å) | (%), ≧25% |
| 5.5 | 16.07 | 100 |
| 12.9 | 6.84 | 62 |
| 13.4 | 6.61 | 35 |
| 16.5 | 5.38 | 37 |
| 19.9 | 4.47 | 69 |
| 20.1 | 4.42 | 26 |
| 21.4 | 4.15 | 37 |
| 21.9 | 4.06 | 77 |
| 30.6 | 2.92 | 25 |

The crystalline hydrobromide salt of the compound of formula (I), unsolvated Form C, may be characterized by its X-ray diffraction pattern, comprising the peaks:

TABLE 4

| | | Relative Intensity |
|---|---|---|
| ANGLE °2θ | d-Spacing (Å) | (%), ≧25% |
| 16.1 | 5.50 | 32 |
| 17.4 | 5.10 | 37 |
| 18.6 | 4.76 | 37 |
| 19.8 | 4.48 | 37 |
| 20.2 | 4.40 | 30 |
| 21.4 | 4.14 | 57 |
| 24.6 | 3.61 | 100 |
| 25.1 | 3.54 | 27 |
| 27.9 | 3.20 | 30 |

The amorphous hydrobromide salt of the compound of formula (I) may be characterized by an X-ray diffraction pattern which lacks distinct narrow peaks. More particularly, amorphous Form D may be characterized by a broad peak centered at an angle 2θ of about 26 degrees.

The crystalline, anhydrous hydrochloride salt of the compound of formula (I), may be characterized by its X-ray diffraction pattern, comprising the peaks:

TABLE 5

| | | Relative Intensity |
|---|---|---|
| ANGLE °2θ | d-Spacing (Å) | (%) |
| 6.603 | 13.375 | 33.5 |
| 7.188 | 12.288 | 27.6 |
| 8.854 | 9.979 | 18.5 |
| 10.688 | 8.271 | 21.3 |
| 12.504 | 7.073 | 20.9 |
| 13.259 | 6.672 | 39.0 |
| 13.906 | 6.363 | 15.0 |
| 15.063 | 5.877 | 18.1 |
| 16.314 | 5.429 | 13.0 |
| 16.755 | 5.287 | 21.3 |
| 17.095 | 5.182 | 30.3 |
| 17.917 | 4.947 | 100.0 |
| 19.734 | 4.495 | 37.0 |
| 20.102 | 4.414 | 59.4 |
| 20.579 | 4.312 | 28.0 |
| 20.973 | 4.232 | 44.1 |
| 21.711 | 4.090 | 21.7 |
| 22.014 | 4.034 | 12.6 |
| 22.632 | 3.926 | 33.9 |
| 24.349 | 3.653 | 21.7 |
| 25.260 | 3.523 | 19.7 |
| 25.419 | 3.501 | 15.7 |
| 25.678 | 3.466 | 29.1 |
| 26.308 | 3.385 | 10.2 |
| 26.816 | 3.322 | 26.4 |
| 27.126 | 3.285 | 24.8 |
| 27.826 | 3.203 | 11.4 |
| 28.094 | 3.174 | 21.3 |
| 28.422 | 3.138 | 18.9 |
| 28.683 | 3.110 | 10.2 |
| 30.684 | 2.9114 | 15.7 |
| 34.335 | 2.6096 | 10.6 |

The crystalline di-hydrate hydrochloride salt of the compound of formula (I), may be characterized by its X-ray diffraction pattern, comprising the peaks:

TABLE 6

| | | Relative Intensity |
|---|---|---|
| ANGLE °2θ | d-Spacing (Å) | (%) |
| 5.519 | 15.999 | 78.2 |
| 10.105 | 8.746 | 42.0 |
| 10.667 | 8.286 | 33.3 |
| 10.933 | 8.086 | 100.0 |
| 12.266 | 7.210 | 54.3 |
| 13.583 | 6.513 | 83.5 |
| 13.864 | 6.382 | 22.4 |
| 14.769 | 5.993 | 37.4 |
| 16.075 | 5.509 | 18.9 |
| 16.396 | 5.402 | 39.9 |
| 20.057 | 4.423 | 52.3 |
| 20.248 | 4.382 | 48.6 |
| 20.491 | 4.331 | 55.4 |
| 21.835 | 4.067 | 58.0 |
| 22.105 | 4.018 | 55.6 |
| 22.800 | 3.897 | 23.2 |
| 23.264 | 3.820 | 21.2 |
| 23.859 | 3.726 | 23.7 |
| 24.600 | 3.616 | 41.8 |
| 24.808 | 3.586 | 23.5 |
| 25.742 | 3.458 | 38.9 |
| 27.481 | 3.243 | 25.3 |
| 28.696 | 3.108 | 31.3 |
| 29.050 | 3.071 | 29.8 |
| 29.416 | 3.034 | 15.8 |
| 30.205 | 2.9564 | 15.2 |
| 30.602 | 2.9189 | 25.5 |
| 30.963 | 2.8857 | 17.9 |
| 31.534 | 2.8348 | 17.1 |
| 32.179 | 2.7794 | 17.3 |
| 32.577 | 2.7463 | 17.3 |
| 32.971 | 2.7144 | 23.3 |
| 33.731 | 2.6550 | 13.4 |
| 34.576 | 2.5920 | 20.0 |

The crystalline hydroiodide salt of the compound of formula (I) may be characterized by its X-ray diffraction pattern, comprising the peaks:

TABLE 7

| | | Relative Intensity |
|---|---|---|
| ANGLE °2θ | d-Spacing (Å) | (%) |
| 6.176 | 14.299 | 96 |
| 7.946 | 11.117 | 48.3 |
| 9.334 | 9.467 | 41.5 |
| 10.803 | 8.183 | 49.4 |
| 13.926 | 6.354 | 43.8 |
| 15.783 | 5.61 | 67.6 |
| 16.277 | 5.441 | 69.9 |
| 16.995 | 5.213 | 79.5 |
| 18.021 | 4.918 | 75.6 |
| 18.399 | 4.818 | 33.5 |
| 18.742 | 4.731 | 84.7 |
| 19.712 | 4.5 | 43.8 |
| 19.977 | 4.441 | 59.1 |
| 21.787 | 4.076 | 88.1 |
| 22.472 | 3.953 | 39.8 |
| 23.22 | 3.827 | 57.4 |
| 23.452 | 3.79 | 100 |
| 23.74 | 3.745 | 47.2 |
| 24.113 | 3.688 | 51.7 |
| 24.442 | 3.639 | 34.1 |
| 24.704 | 3.601 | 72.2 |
| 25.15 | 3.538 | 48.9 |
| 25.594 | 3.478 | 46 |
| 26.452 | 3.367 | 69.3 |
| 26.919 | 3.309 | 50 |
| 27.294 | 3.265 | 36.9 |
| 27.717 | 3.216 | 42 |
| 28.257 | 3.156 | 44.3 |
| 28.943 | 3.082 | 21.6 |
| 29.814 | 2.9943 | 42.6 |
| 30.124 | 2.9642 | 32.4 |
| 31.423 | 2.8445 | 20.5 |
| 32.102 | 2.7859 | 36.4 |
| 33.276 | 2.6902 | 29.5 |
| 33.666 | 2.66 | 17.6 |
| 34.143 | 2.6239 | 19.3 |
| 34.465 | 2.6001 | 28.4 |

The crystalline cyclohexanesulfamate salt of the compound of formula (I) may be characterized by its X-ray diffraction pattern, comprising the peaks:

TABLE 8

| | | Relative Intensity |
|---|---|---|
| ANGLE °2θ | d-Spacing (Å) | (%) |
| 5.589 | 15.800 | 28.5 |
| 10.611 | 8.330 | 27.7 |
| 11.119 | 7.951 | 20.4 |
| 11.324 | 7.808 | 32.2 |
| 13.202 | 6.700 | 10.9 |
| 13.611 | 6.500 | 10.4 |
| 14.856 | 5.958 | 10.4 |
| 15.140 | 5.847 | 11.3 |
| 16.327 | 5.425 | 48.6 |
| 16.651 | 5.320 | 26.6 |
| 17.280 | 5.128 | 20.6 |
| 17.745 | 4.994 | 31.7 |
| 18.513 | 4.789 | 25.9 |
| 19.364 | 4.580 | 100.0 |
| 19.792 | 4.482 | 14.7 |
| 20.176 | 4.398 | 53.0 |
| 20.935 | 4.240 | 55.3 |
| 21.233 | 4.181 | 33.8 |
| 22.312 | 3.981 | 16.7 |
| 22.637 | 3.925 | 66.1 |
| 23.038 | 3.857 | 14.2 |
| 23.608 | 3.765 | 47.3 |
| 24.051 | 3.697 | 29.1 |
| 24.339 | 3.654 | 11.0 |
| 25.304 | 3.517 | 13.3 |
| 25.976 | 3.427 | 16.0 |
| 26.504 | 3.360 | 11.6 |
| 27.148 | 3.282 | 23.3 |
| 28.436 | 3.136 | 16.5 |
| 28.825 | 3.095 | 20.7 |
| 29.299 | 3.046 | 10.3 |
| 29.600 | 3.015 | 14.9 |
| 29.923 | 2.984 | 23.7 |
| 31.264 | 2.859 | 10.8 |
| 32.236 | 2.775 | 17.0 |

The crystalline mono-hydrate sulfate salt of the compound of formula (I) may be characterized by its X-ray diffraction pattern, comprising the peaks:

TABLE 9

| | | Relative Intensity |
|---|---|---|
| ANGLE °2θ | d-Spacing (Å) | (%) |
| 5.349 | 16.508 | 100 |
| 11.725 | 7.541 | 24.1 |
| 14.141 | 6.258 | 8.6 |
| 15.764 | 5.617 | 17.1 |
| 18.052 | 4.91 | 31.9 |
| 18.483 | 4.796 | 7.8 |
| 18.9 | 4.691 | 8.9 |
| 19.124 | 4.637 | 20.1 |
| 20.303 | 4.37 | 17 |
| 20.626 | 4.303 | 9.5 |
| 21.142 | 4.199 | 10.6 |
| 21.709 | 4.09 | 23 |
| 22.269 | 3.989 | 6.2 |
| 22.854 | 3.888 | 16.2 |
| 23.149 | 3.839 | 9.9 |
| 23.629 | 3.762 | 8.3 |
| 24.92 | 3.57 | 5.2 |
| 25.218 | 3.529 | 6.6 |
| 25.76 | 3.456 | 5 |
| 26.033 | 3.42 | 6.2 |
| 26.325 | 3.383 | 16.1 |
| 28.097 | 3.173 | 7.8 |
| 28.949 | 3.082 | 6.2 |
| 29.974 | 2.9787 | 6.7 |

The following examples describe the invention in greater detail and are intended to illustrate the invention, but not to limit it.

### EXAMPLE 1

### Preparation of N-{2-[4-(2-(2-propoxy)phenyl)-1-piperazinyl]ethyl}-2-(2-oxo-piperidin-1-yl)acetamide Hydrobromide, Form A

Step A: Formation of 1-(ethoxycarbonylmethyl)-2-piperidone

Ethyl bromoacetate (93.2 g, 558 mmol) was mixed with 2-piperidone (54.2 g, 546.8 mmol) and toluene (60.0 g). The solution was then cooled to a temperature of about 5-15° C.

In a separate addition funnel was placed a suspension of NaH (60% in oil) (22.3 g, 583.3 mmol) in toluene (108.5 g). The stirred NaH suspension was added to the above solution in sequential small portions with cooling to maintain the reaction temperature at about 5-15° C. (The reaction is highly exothermic.) At the end of addition, the mixture formed a slightly yellowish solution which then became a beige suspension.

The reaction mixture was poured onto a solution of ammonium chloride (30.0 g) in purified water (115.0 g). The resulting phases were separated. The organic phase was re-extracted with a solution of sodium chloride (20.0 g) in water. The 1^{st} aqueous phase was re-extracted with toluene (3×100 g). The organic phases were separately concentrated to oils. The oils were combined and dissolved, with heating, in tert-butyl methyl ether (75.0 g), seeded with product and cooled to about 20° C. To the resulting solution was added cyclohexane (75.0 g) and the mixture was allowed to stand overnight, resulting in formation of a precipitate. The mixture was then cooled to about 0-5° C. and stirred for 1 hour. The precipitate was filtered and washed with a cold mixture of tert-butyl methyl ether (20.0 g) and cyclohexane (20.0 g). The wet product was dried under vacuum at about 40° C. to yield the solid product.

Yield: 84.6 g (83.5%)

Step B: Formation of N-{2-[4-(2-(2-propoxy)phenyl)-1-piperanzinyl]ethyl}-2-(2-oxo-piperidin-1-yl)acetamide

1-(ethoxycarbonylmethyl)-2-piperidone (44.98 g, 0.241 mole) from Step A was added to purified water (134.20 g) with stirring until completely dissolved. To the resulting solution was added a solution of sodium hydroxide (21.20 g, 0.530 mole) in purified water (44.80 g). The solution temperature was maintained below about 30° C. Following complete addition of the sodium hydroxide solution, the resulting mixture was allowed to stand for 30 min, resulting in a clear yellow solution.

To the solution was then added 4-[2-(1-methylethoxy)phenyl]-1-piperazine ethanamine .3HBr (101.2 g, 0.199 mol). The mixture was stirred at a temperature of less than about 6° C. until the HBr salt was completely dissolved. To the resulting mixture was added a solution of DCC (52.00 g, 0.250 mole) in isopropanol (180.2 g). The resulting mixture was heated to about 50-60° C., resulting in a beige suspension, which darkened with time. The mixture was stirred for about 30 min then cooled to about 0-10° C. The precipitate was filtered and washed with purified water (179.0 g) until the wet filter cake turned white. The clear brown filtrate and washes were combined and heated under vacuum to a temperature of about 55° C. to azeotropically remove the isopropanol. When about ⅓ of the filtrate volume was distilled off, the clear brown solution became an emulsion. Toluene (270.0 g) and a solution of sodium hydroxide (12.0 g) in purified water (28.0 g) were added. The resulting mixture was heated to about 55-60° C., stirred for 30 min and let stand to form two separate layers. The aqueous layer was discarded. The organic (toluene) layer was extracted with a 10% aqueous sodium chloride solution (60.0 g). The organic phase was separated and concentrated under vacuum at a temperature of less than 55° C. to yield the crude free base product as an oil.

Step C: Formation of Hydrobromide Salt

The crude oil product from Step B (96.6 g, 240 mmol) was dissolved in anhydrous ethanol (denatured with 1% toluene) (250.0 g) and concentrated under vacuum at a temperature of less than 55° C. to azeotropically remove any water. To the residue was added anhydrous ethanol (denatured with 1% toluene) (235.0 g), the solution was cooled to about 5° C. and filtered to remove any insoluble impurities. The resulting solution was seeded with the desired hydrobromide salt (0.20 g) and then charged with hydrogen bromide gas (15.30 g, 0.189 mol). The resulting mixture was stirred at about 0-5° C. for 1 h, resulting in crystallization of the hydrobromide salt product which was collected by filtration. The wet solid product was slurried at about 50-60° C. for 30 min in anhydrous ethanol (denatured with 1% toluene) (150.0 g) then cooled to about 0-5° C. The precipitate was filtered and washed with anhydrous ethanol (denature with 1% toluene) (30.0 g). The wet product was dried under vacuum at less than about 60° C. to yield the product as a slightly yellowish solid.

Yield: 81.8 g (85%).

### EXAMPLE 2

### Preparation of N-{2-[4-(2-(2-propoxy)phenyl)-1-piperanzinyl]ethyl}-2-(2-oxo-piperidin-1-yl)acetamide Hydrobromide, Form A

N-[2-[4-[2-(1-methylethoxy)phenyl]-1-piperazinyl]ethyl]-2-oxo-1-piperidineacetamide (2.49 g, 6.13 mmol) was dissolved in ethyl acetate (10 mL). To the solution were added methanol (0.5 mL) and a solution of 0.9N HBr in ethyl acetate (6.87 mL, 6.19 mmol). Soft solid lumps were observed to form first, followed by the formation of a white solid. More methanol was added to fully dissolve the white solid. The solution was cooled, the resulting precipitate was collected by filtration and washed with ethyl acetate to yield the product as a white solid.

| | |
|---|---|
| m.p. | 204-207° C. |
| Water (KF): | 0.29% |
| Elemental analysis for | |
| C₂₂H₃₅BrN₄O₃: | |
| Calculated: | C 54.66; H 7.30; Br 16.53; N 11.59 |
| Found: | C 54.67; H 7.29; Br 16.42; N 11.56 |

### EXAMPLE 3

### Preparation of N-{2-[4-(2-(2-propoxy)phenyl)-1-piperazinyl]ethyl}-2-(2-oxo-piperidin-1-yl)acetamide Cylcohexanesulfamate

N-[2-[4-[2-(1-methylethoxy)phenyl]-1-piperazinyl]ethyl]-2-oxo-1-piperidineacetamide (36.2 g, 90 mmol) was dissolved in ethyl acetate (200 mL)and treated with a solution of cyclamic acid (16.13 g, 90 mmol) in methanol. The resulting solution was heated on a steam bath to remove some of the methanol. The solution was allowed to stand at room temperature and the solid product crystallized out. Petroleum ether was added to precipitate more product. The precipitate was collected by filtration and air-dried to yield the product as a solid.

Yield: 34.2 g (65%)

A sample of the collected product (3.28 g) was recrystallized by dissolving it in ethyl acetate (30 mL) and methanol (10 mL) and then evaporating some of the methanol by heating. The precipitated crystalline solid was collected by filtration to yield the solid product.

Yield: 2.77 g (2 crops, 84%)
mp 139-141° C.
Water (KF): 0.13%
Elemental analysis for C₂₈H₄₇N₅O₆S:

| | |
|---|---|
| Calculated: | C 57.81; H 8.14; N 12.04; S 5.51 |
| Found: | C 57.73; H 8.17; N 11.98; S 5.27. |

### EXAMPLE 4

### Preparation of N-{2-[4-(2-(2-propoxy)phenyl)-1-piperazinyl]ethyl}-2-(2-oxo-piperidin-1-yl)acetamide Sulfate

N-{2-[4-(2-(2-propoxy)phenyl)-1-piperazinyl]ethyl}-2-(2-oxo-piperidin-1-yl)acetamide (5.80 g, 14.4 mmol) was dissolved in ethyl acetate on a steam bath. The solution was cooled to ambient temperature. Concentrated sulfuric acid (1.44 g, 14.4 mmol) was then added dropwise to the solution, with stirring, resulting in the formation of lumps of sticky solid material. Methanol (10 mL) was added to the mixture, and the mixture was then heated on a steam bath until all the solid lumps dissolved. The resulting solution was cooled, seeded with the desired H₂SO₄ salt (0.1 g) and stirred at room temperature. The solution was stirred overnight, the solid precipitate was filtered, washed with ethyl acetate and air-dried to yield the product as a pale yellow solid.

Yield: 5.42 g (73%)
m.p. 102.2-105.1° C.
Water (KF): calculated: 3.48%, found: 4.11%
Elemental analysis for C₂₂H₃₈N₄O₈S:

| | |
|---|---|
| Calculated: | C 50.95; H 7.39; N 10.80; S 6.18 |
| Found: | C 50.43; H 7.26; N 10.62; S 6.49 |

### EXAMPLE 5

### Preparation of N-{2-[4-(2-(2-propoxy)phenyl)-1-piperazinyl]ethyl}-2-(2-oxo-piperidin-1-yl) acetamide hydrochloride dihydrate

N-{2-[4-(2-(2-propoxy) phenyl)-1-piperazinyl]ethyl}-2-(2-oxo-piperidin-1-yl)acetamide (147.0 g, 0.322 mol) was dissolved in ethyl acetate (700 mL), methanol (40 mL) and H₂O (13.2 mL). The mixture was warmed on a steam bath to dissolve the free base then cooled to room temperature. To the resulting solution was added 2.25 M HCl in ethyl acetate (162 mL, 0.364 mol), dropwise with continuous stirring. A solid product was formed slowly at the beginning and then became a thick slurry after addition of all of the HCl solution. The slurry was stirred at room temperature for 2 hours, the product was collected by filtration, washed with ethyl acetate (200 mL) and air-dried to yield the product as a white solid.

Yield: 116.0 g (76%)

mp: 83-85° C.

### EXAMPLE 6

### Preparation of N-{2-[4-(2-(2-propoxy)phenyl)-1-piperazinyl]ethyl}-2-(2-oxo-piperidin-1-yl)acetamide hydrochloride as a Mixture of Anhydrous and Dihydrate Forms

A slurry of N-{2-[4-(2-(2-propoxy)phenyl)-1-piperazinyl]ethyl}-2-(2-oxo-piperidin-1-yl)acetamide hydrochloride dihydrate (313.0 g, 0.659 mol) in ethyl acetate (1.8 L) was heated to reflux. The ethyl acetate was distilled to remove excess water as an azeotropic mixture of ethyl acetate/H₂O (bp 63° C.) using a Dean-Stark trap. The volume of the mixture was maintained constant by adding ethyl acetate from a 500 mL addition funnel. When the vapor temperature reached 75° C., distillation was stopped and the reaction mixture was cooled to room temperature. The tan-colored solid was collected by filtration, washed with ethyl acetate (0.2 L) and air-dried. The solid was further dried in a vacuum oven at room temperature for 72 h to yield the solid product.

mp: 184-186° C.
Water (KF): 4.11%
Elemental analysis for C₂₂H₃₇ClN₄O₄:

| | |
|---|---|
| Calculated: | C 57.82; H 8.17; N 12.26; Cl 7.76 |
| Found: | C 57.94; H 8.14; N 12.17; Cl 7.73 |

### EXAMPLE 7

### Preparation of N-{2-[4-(2-(2-propoxy)phenyl)-1-piperazinyl]ethyl}-2-(2-oxo-piperidin-1-yl)acetamide hydrochloride Anhydrous Form

A slurry of N-{2-[4-(2-(2-propoxy)phenyl)-1-piperazinyl]ethyl}-2-(2-oxo-piperidin-1-yl)acetamide hydrochloride dihydrate (2.0 g) in xylene (20 mL) was heated at reflux (137-141° C.) with stirring for 30 min. The mixture was cooled to room temperature and the solid was collected by filtration and air-dried.

Yield: 1.67 g (90%)

mp 186-188° C.

Water (KF): 0.39%.

### EXAMPLE 8

### Preparation of N-{2-[4-(2-(2-propoxy)phenyl)-1-piperazinyl]ethyl}-2-(2-oxo-piperidin-1-yl)acetamide hydrochloride Anhydrous Form

N-{2-[4-(2-(2-propoxy)phenyl)-1-piperazinyl]ethyl}-2-(2-oxo-piperidin-1-yl)acetamide hydrochloride di-hydrate (50 mg) was heated in a powder x-ray diffraction hot stage to 140° C. and then cooled to 25° C., resulting in the anhydrous form.

m.p. 189-191° C.

### EXAMPLE 9

### Preparation of N-{2-[4-(2-(2-propoxy)phenyl)-1-piperazinyl]ethyl}-2-(2-oxo-piperidin-1-yl)acetamide Hydroiodide

N-{2-[4-(2-(2-propoxy)phenyl)-1-piperazinyl]ethyl}-2-(2-oxo-piperidin-1-yl)acetamide (5.12 g, 12.7 mmol) was dissolved in ethyl acetate on a steam bath. HI (3.46 g, 12.7 mmol) was added to the solution resulting in a nearly clear solution. The solvent was evaporated under reduced pressure. Ethyl acetate was added to the residue and the mixture was heated on a steam bath. Methanol (10 mL) was added to the suspension and the mixture was heated at about 40-50° C. The resulting mixture was then cooled to room temperature with stirring over about 30 min, resulting in the formation of a precipitate. The precipitate was collected by filtration and washed with ethyl acetate to yield the solid product as an off-white solid. The solid was immediately transferred to a brown bottle (product is light sensitive) and dried overnight under vacuum at room temperature.

Yield: 4.55 g (67%)
m.p. 201.0-202.9° C.
Water (KF): 0.22%
Elemental analysis for C₂₂H₃₅IN₄O₃:

| | |
|---|---|
| Calculated: | C 49.81; H 6.65; I 23.92; N 10.56 |
| Found: | C 49.70; H 6.65; I 23.99; N 10.36 |

### EXAMPLE 10

### Preparation of N-{2-[4-(2-(2-propoxy)phenyl)-1-piperazinyl]ethyl}-2-(2-oxo-piperidin-1-yl)acetamide Hydrobromide, Amorphous Form D

Form A of the hydrobromide salt, prepared as described above (about 30 mg) was dissolved in dichloromethane (1 mL). The solution was filtered through a 0.2 μm nylon filter into a round bottom flask and the solvent removed by rotary evaporation. The sample was then placed in a vacuum oven at room temperature and dried overnight, to yield the title product.

### EXAMPLE 11

### Preparation of N-{2-[4-(2-(2-propoxy)phenyl)-1-piperazinyl]ethyl}-2-(2-oxo-piperidin-1-yl)acetamide Hydrobromide, Amorphous Form D

Form A of the hydrobromide salt, prepared as described above (about 30 mg) was dissolved in methanol (1 mL). The solution was filtered through a 0.2 μm nylon filter into a round bottom flask and the solvent removed by rotary evaporation. The sample was then placed in a vacuum oven at room temperature and dried overnight, to yield the title product.

### EXAMPLE 12

### Preparation of N-{2-[4-(2-(2-propoxy)phenyl)-1-piperazinyl]ethyl}-(2-oxo-piperidin-1-yl)acetamide Hydrobromide, Form C

Form D of the hydrobromide salt (about 30 mg), prepared as above, was placed in an oven at a temperature of about 100° C. for about 3 hours. The sample was then removed from the oven and allowed to cool to room temperature to yield the title compound.

### EXAMPLE 13

### Preparation of N-{2-[4-(2-(2-propoxy)phenyl)-1-piperazinyl]ethyl}-2-(2-oxo-piperidin-1-yl)acetamide Hydrobromide, Form B

Form A of the hydrobromide salt, prepared as described above (about 30 mg) was dissolved in methanol (1 mL) with sonication. The solution was filtered through a 0.2 μm nylon filter to yield a clear solution. The solution was allowed to evaporate uncovered at room temperature to yield the title compound.

### EXAMPLE 14

### Preparation of N-{2-[4-(2-(2-propoxy)phenyl)-1-piperazinyl]ethyl}-2-(2-oxo-piperidin-1-yl)acetamide Hydrobromide, Form B

Form A of the hydrobromide salt, prepared as described above (about 30 mg) was dissolved in about water (1 mL) with sonication. The solution was filtered through a 0.2 μm nylon filter to yield a clear solution. The vial containing the solution was covered with foil containing several pinholes and the solution was allowed to evaporate at room temperature, to yield the title compound.

### EXAMPLE 15

### Preparation of N-{2-[4-(2-2-propoxy)phenyl)1-piperazinyl]ethyl}-2-(2-oxo-piperidin-1-yl)acetamide Hydrobromide, Form A

Methanol (0.5 mL) was added to a vial and heated to about 60° C. on a hot plate. Form A of the hydrobromide salt, prepared as above, was added to form a supersaturated solution. The solution was then filtered through a 0.2 μm nylon filter into a heated vial. The heat source was turned off and the solution was allowed to slowly cool to room temperature overnight. The resulting solids were recovered by vacuum filtration and air-dried at room temperature to yield the title compound.

### EXAMPLE 16

### Preparation of N-{2-[4-(2-(2-propoxy)phenyl)-1-piperazinyl]ethyl}-2-(2-oxo-piperidin-1-yl)acetamide Hydrobromide, Form A

Form A of the hydrobromide salt; prepared as above (about 15 mg) were dissolved in N,N-dimethylformamide (1 mL) with sonication. The solution was filtered through a 0.2 μm nylon filter to yield a clear solution. The solution was allowed to evaporate uncovered. The resulting solid was collected to yield the title compound.

### EXAMPLE 17

### Preparation of N-{2-[4-(2-(2-propoxy)phenyl)-1-piperazinyl]ethyl}-2-(2-oxo-piperidin-1-yl)acetamide Hydrobromide, Form A

Ethanol (0.5 mL) was added to a vial and heated to about 60° C. on a hot plate. Form A of the hydrobromide salt, prepared as above, was added to form a supersaturated solution. The solution was filtered through a 0.2 μm nylon filter into a vial containing ethyl acetate (10 mL) cooled in an acetone/dry ice bath (at about −78° C.) and stirred for approximately 10 minutes. The resulting solids were recovered by vacuum filtration and air-dried at room temperature, to yield the title compound.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for preparing a solid hydrobromide salt of the compound of formula (I) 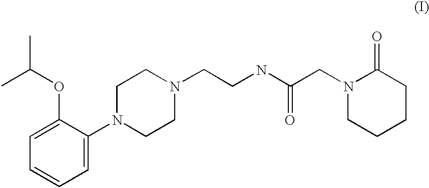
comprising
reacting the compound of formula (I) with hydrogen bromide gas, in a polar organic solvent.

2. The process of claim 1 wherein the hydrogen bromide gas is present in an amount in the range of about 0.95 to about 1.05 molar equivalents.

3. A process for preparing a solid hydrochloride salt of the compound of formula (I) 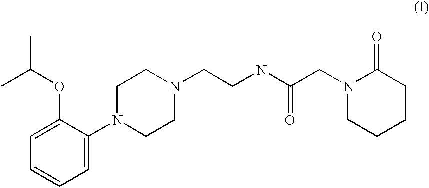
comprising
reacting the compound of formula (I) with hydrogen chloride gas, in a polar organic solvent.

4. The process of claim 3, wherein the hydrogen chloride gas is present in an amount in the range of about 0.95 to 1.05 molar equivalents.

5. A process for preparing a solid hydroiodide salt of the compound of formula (I) 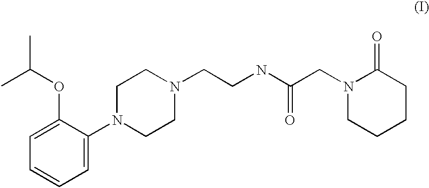
comprising
reacting the compound of formula (I) with concentrated hydroiodic acid, in a polar organic solvent.

6. The process of claim 5, wherein the hydroiodic acid is present in an amount in the range of about 0.95 to 1.05 molar equivalents.

7. A process for preparing a solid cyclohexanesulfamate salt of the compound of formula (I) 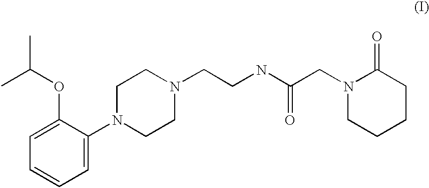
comprising
reacting the compound of formula (I) with cyclohexanesulfamic acid, in a polar organic solvent.

8. The process of claim 7 wherein the cyclohexanesulfamic acid is present in an amount in the range of about 0.95 to about 1.05 molar equivalents.

9. A process for preparing a solid sulfate salt of the compound of formula (I) 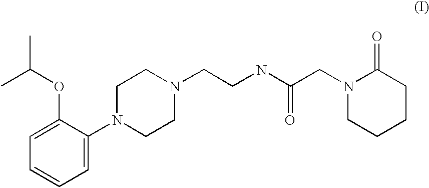
comprising
reacting the compound of formula (I) with concentrated sulfuric acid, in a polar organic solvent.

10. The process of claim 9 wherein the sulfuric acid is present in an amount in the range of about 0.95 to about 1.05 molar equivalents.

11. A solid salt of N-{2-[4-(2-(2-propoxy)phenyl)-1-piperazinyl]ethyl}-2-(2-oxo-piperidin-1-yl)acetamide.

12. A solid hydrobromide salt of N-{2-[4-(2-(2-propoxy)phenyl)-1-piperazinyl]ethyl}-2-(2-oxo-piperidin-1-yl)acetamide.

13. The solid salt as in claim 12, wherein the salt is a mono-hydrobromide salt.

14. The solid salt as in claim 13, wherein the salt is an unsolvated mono-hydrobromide salt.

15. A crystalline hydrobromide salt of N-{2-[4-(2-(2-propoxy)phenyl)-1-piperazinyl]ethyl}-2-(2-oxo-piperidin-1-yl)acetamide comprising the following X-ray diffraction peaks:

| | | Relative |
|---|---|---|
| ANGLE °2θ | d-Spacing (Å) | Intensity (%) |
| 6.583 | 13.416 | 65.7 |
| 7.293 | 12.111 | 29.2 |
| 8.919 | 9.906 | 14 |
| 12.208 | 7.244 | 15 |
| 13.039 | 6.784 | 47.8 |
| 13.624 | 6.494 | 17.1 |
| 13.988 | 6.326 | 11.9 |
| 14.867 | 5.954 | 15.2 |
| 16.343 | 5.419 | 25.9 |
| 16.781 | 5.279 | 10.2 |
| 17.189 | 5.154 | 33.3 |
| 18.006 | 4.922 | 100 |
| 19.354 | 4.582 | 24.9 |
| 20.067 | 4.421 | 66.7 |
| 20.32 | 4.367 | 23.7 |
| 20.884 | 4.25 | 47.4 |
| 21.131 | 4.201 | 27.6 |
| 21.545 | 4.121 | 20.1 |
| 21.939 | 4.048 | 46.1 |
| 22.665 | 3.92 | 39.1 |
| 23.663 | 3.757 | 16.7 |
| 24.272 | 3.664 | 28.7 |
| 24.599 | 3.616 | 53.1 |
| 25.006 | 3.558 | 40.8 |
| 25.379 | 3.507 | 17.4 |
| 26.372 | 3.377 | 29.7 |
| 26.755 | 3.329 | 62.6 |
| 27.482 | 3.243 | 23 |
| 27.85 | 3.201 | 59 |
| 28.636 | 3.115 | 12.8 |
| 29.54 | 3.021 | 10.4 |
| 29.722 | 3.003 | 15.9 |
| 30.354 | 2.9422 | 21.7 |
| 31.18 | 2.8662 | 13.5 |
| 31.637 | 2.8257 | 13 |
| 31.956 | 2.7983 | 11.9 |
| 33.003 | 2.7119 | 11.3 |
| 33.334 | 2.6857 | 14.8 |
| 34.698 | 2.5832 | 10.6 |

16. The solid salt as in claim 13, wherein the salt is a solvated mono-hydrobromide salt.

17. A crystalline hydrobromide salt of N-{2-[4-(2-(2-propoxy)phenyl)-1-piperazinyl]ethyl}-2-(2-oxo-piperidin-1-yl)acetamide comprising the following X-ray diffraction peaks:

| | | Relative |
|---|---|---|
| ANGLE °2θ | d-Spacing (Å) | Intensity (%) |
| 5.5 | 16.07 | 100 |
| 12.9 | 6.84 | 62 |
| 13.4 | 6.61 | 35 |
| 16.5 | 5.38 | 37 |
| 19.9 | 4.47 | 69 |
| 20.1 | 4.42 | 26 |
| 21.4 | 4.15 | 37 |
| 21.9 | 4.06 | 77 |
| 30.6 | 2.92 | 25 |

18. A crystalline hydrobromide salt of N-{2-[4-(2-(2-propoxy)phenyl)-1-piperazinyl]ethyl}-2-(2-oxo-piperidin-1-yl)acetamide comprising the following X-ray diffraction peaks:

| | | Relative |
|---|---|---|
| ANGLE °2θ | d-Spacing (Å) | Intensity (%) |
| 16.1 | 5.50 | 32 |
| 17.4 | 5.10 | 37 |
| 18.6 | 4.76 | 37 |
| 19.8 | 4.48 | 37 |
| 20.2 | 4.40 | 30 |
| 21.4 | 4.14 | 57 |
| 24.6 | 3.61 | 100 |
| 25.1 | 3.54 | 27 |
| 27.9 | 3.20 | 30 |

19. A solid hydrochloride salt of N-{2-[4-(2-(2-propoxy)phenyl)-1-piperazinyl]ethyl}-2-(2-oxo-piperidin-1-yl)acetamide.

20. The solid salt as in claim 19, wherein the salt is a mono-hydrochloride salt.

21. The solid salt as in claim 20, wherein the salt is an anhydrous mono-hydrochloride salt.

22. A crystalline hydrochloride salt of N-{2-[4-(2-(2-propoxy)phenyl)-1-piperazinyl]ethyl}-2-( 2-oxo-piperidin-1-yl)acetamide comprising the following X-ray diffraction peaks:

| | | Relative |
|---|---|---|
| ANGLE °2θ | d-Spacing (Å) | Intensity (%) |
| 6.603 | 13.375 | 33.5 |
| 7.188 | 12.288 | 27.6 |
| 8.854 | 9.979 | 18.5 |
| 10.688 | 8.271 | 21.3 |
| 12.504 | 7.073 | 20.9 |
| 13.259 | 6.672 | 39.0 |
| 13.906 | 6.363 | 15.0 |
| 15.063 | 5.877 | 18.1 |
| 16.314 | 5.429 | 13.0 |
| 16.755 | 5.287 | 21.3 |
| 17.095 | 5.182 | 30.3 |
| 17.917 | 4.947 | 100.0 |
| 19.734 | 4.495 | 37.0 |
| 20.102 | 4.414 | 59.4 |
| 20.579 | 4.312 | 28.0 |
| 20.973 | 4.232 | 44.1 |
| 21.711 | 4.090 | 21.7 |
| 22.014 | 4.034 | 12.6 |
| 22.632 | 3.926 | 33.9 |
| 24.349 | 3.653 | 21.7 |
| 25.260 | 3.523 | 19.7 |
| 25.419 | 3.501 | 15.7 |
| 25.678 | 3.466 | 29.1 |
| 26.308 | 3.385 | 10.2 |
| 26.816 | 3.322 | 26.4 |
| 27.126 | 3.285 | 24.8 |
| 27.826 | 3.203 | 11.4 |
| 28.094 | 3.174 | 21.3 |
| 28.422 | 3.138 | 18.9 |
| 28.683 | 3.110 | 10.2 |
| 30.684 | 2.9114 | 15.7 |
| 34.335 | 2.6096 | 10.6 |

23. The solid salt as in claim 20, wherein the salt is a di-hydrate mono-hydrochloride salt.

24. A crystalline form hydrochloride salt of N-{2-[4-(2-(2-propoxy)phenyl)-1-piperazinyl]ethyl}-2-(2-oxo-piperidin-1-yl)acetamide comprising the following X-ray diffraction peaks:

| | | Relative |
|---|---|---|
| ANGLE °2θ | d-Spacing (Å) | Intensity (%) |
| 5.519 | 15.999 | 78.2 |
| 10.105 | 8.746 | 42.0 |
| 10.667 | 8.286 | 33.3 |
| 10.933 | 8.086 | 100.0 |
| 12.266 | 7.210 | 54.3 |
| 13.583 | 6.513 | 83.5 |
| 13.864 | 6.382 | 22.4 |
| 14.769 | 5.993 | 37.4 |
| 16.075 | 5.509 | 18.9 |
| 16.396 | 5.402 | 39.9 |
| 20.057 | 4.423 | 52.3 |
| 20.248 | 4.382 | 48.6 |
| 20.491 | 4.331 | 55.4 |
| 21.835 | 4.067 | 58.0 |
| 22.105 | 4.018 | 55.6 |
| 22.800 | 3.897 | 23.2 |
| 23.264 | 3.820 | 21.2 |
| 23.859 | 3.726 | 23.7 |
| 24.600 | 3.616 | 41.8 |
| 24.808 | 3.586 | 23.5 |
| 25.742 | 3.458 | 38.9 |
| 27.481 | 3.243 | 25.3 |
| 28.696 | 3.108 | 31.3 |
| 29.050 | 3.071 | 29.8 |
| 29.416 | 3.034 | 15.8 |
| 30.205 | 2.9564 | 15.2 |
| 30.602 | 2.9189 | 25.5 |
| 30.963 | 2.8857 | 17.9 |
| 31.534 | 2.8348 | 17.1 |
| 32.179 | 2.7794 | 17.3 |
| 32.577 | 2.7463 | 17.3 |
| 32.971 | 2.7144 | 23.3 |
| 33.731 | 2.6550 | 13.4 |
| 34.576 | 2.5920 | 20.0 |

25. A solid hydroiodide salt of N-{2-[4-(2-(2-propoxy)phenyl)-1-piperazinyl]ethyl}-2-(2-oxo-piperidin-1-yl)acetamide.

26. The solid salt as in claim 25, wherein the salt is a mono-hydroiodide salt.

27. A crystalline hydroiodide salt of N-{2-[4-(2-(2-propoxy)phenyl)-1-piperazinyl]ethyl}-2-(2-oxo-piperidin-1-yl)acetamide comprising the following X-ray diffraction peaks:

| | | Relative |
|---|---|---|
| ANGLE °2θ | d-Spacing (Å) | Intensity (%) |
| 6.176 | 14.299 | 96 |
| 7.946 | 11.117 | 48.3 |
| 9.334 | 9.467 | 41.5 |
| 10.803 | 8.183 | 49.4 |
| 13.926 | 6.354 | 43.8 |
| 15.783 | 5.61 | 67.6 |
| 16.277 | 5.441 | 69.9 |
| 16.995 | 5.213 | 79.5 |
| 18.021 | 4.918 | 75.6 |
| 18.399 | 4.818 | 33.5 |
| 18.742 | 4.731 | 84.7 |
| 19.712 | 4.5 | 43.8 |
| 19.977 | 4.441 | 59.1 |
| 21.787 | 4.076 | 88.1 |
| 22.472 | 3.953 | 39.8 |
| 23.22 | 3.827 | 57.4 |
| 23.452 | 3.79 | 100 |
| 23.74 | 3.745 | 47.2 |
| 24.113 | 3.688 | 51.7 |
| 24.442 | 3.639 | 34.1 |
| 24.704 | 3.601 | 72.2 |
| 25.15 | 3.538 | 48.9 |
| 25.594 | 3.478 | 46 |
| 26.452 | 3.367 | 69.3 |
| 26.919 | 3.309 | 50 |
| 27.294 | 3.265 | 36.9 |
| 27.717 | 3.216 | 42 |
| 28.257 | 3.156 | 44.3 |
| 28.943 | 3.082 | 21.6 |
| 29.814 | 2.9943 | 42.6 |
| 30.124 | 2.9642 | 32.4 |
| 31.423 | 2.8445 | 20.5 |
| 32.102 | 2.7859 | 36.4 |
| 33.276 | 2.6902 | 29.5 |
| 33.666 | 2.66 | 17.6 |
| 34.143 | 2.6239 | 19.3 |
| 34.465 | 2.6001 | 28.4 |

28. A solid cyclohexanesulfamate salt of N-{2-[4-(2-(2-propoxy)phenyl)-1-piperazinyl]ethyl}-2-(2-oxo-piperidin-1-yl)acetamide.

29. The solid salt as in claim 28, wherein the salt is a mono-cyclohexanesulfamate salt.

30. A crystalline cyclohexanesulfamate salt of N-{2-[4-(2-(2-propoxy)phenyl)-1-piperazinyl]ethyl}-2-(2-oxo-piperdin-1-yl)acetamide comprising the following X-ray diffraction peaks:

| | | Relative |
|---|---|---|
| ANGLE °2θ | d-Spacing (Å) | Intensity (%) |
| 5.589 | 15.800 | 28.5 |
| 10.611 | 8.330 | 27.7 |
| 11.119 | 7.951 | 20.4 |
| 11.324 | 7.808 | 32.2 |
| 13.202 | 6.700 | 10.9 |
| 13.611 | 6.500 | 10.4 |
| 14.856 | 5.958 | 10.4 |
| 15.140 | 5.847 | 11.3 |
| 16.327 | 5.425 | 48.6 |
| 16.651 | 5.320 | 26.6 |
| 17.280 | 5.128 | 20.6 |
| 17.745 | 4.994 | 31.7 |
| 18.513 | 4.789 | 25.9 |
| 19.364 | 4.580 | 100.0 |
| 19.792 | 4.482 | 14.7 |
| 20.176 | 4.398 | 53.0 |
| 20.935 | 4.240 | 55.3 |
| 21.233 | 4.181 | 33.8 |
| 22.312 | 3.981 | 16.7 |
| 22.637 | 3.925 | 66.1 |
| 23.038 | 3.857 | 14.2 |
| 23.608 | 3.765 | 47.3 |
| 24.051 | 3.697 | 29.1 |
| 24.339 | 3.654 | 11.0 |
| 25.304 | 3.517 | 13.3 |
| 25.976 | 3.427 | 16.0 |
| 26.504 | 3.360 | 11.6 |
| 27.148 | 3.282 | 23.3 |
| 28.436 | 3.136 | 16.5 |
| 28.825 | 3.095 | 20.7 |
| 29.299 | 3.046 | 10.3 |
| 29.600 | 3.015 | 14.9 |
| 29.923 | 2.984 | 23.7 |
| 31.264 | 2.859 | 10.8 |
| 32.236 | 2.775 | 17.0 |

31. A solid sulfate salt of N-{2-[4-(2-(2-propoxy)phenyl)-1-piperazinyl]ethyl}-2-(2-oxo-piperidin-1-yl)acetamide.

32. The solid salt as in claim 31, wherein the salt is a mono-sulfate salt.

33. The solid salt as in claim 32, wherein the salt is a mono-hydrate mono-sulfate salt.

34. A crystalline sulfate salt of N-{2-[4-(2-(2-propoxy)phenyl)-1-piperazinyl]ethyl}-2-(2-oxo-piperidin-1-yl)acetamide comprising the following X-ray diffraction peaks:

| | | Relative |
|---|---|---|
| ANGLE °2θ | d-Spacing (Å) | Intensity (%) |
| 5.349 | 16.508 | 100 |
| 11.725 | 7.541 | 24.1 |
| 14.141 | 6.258 | 8.6 |
| 15.764 | 5.617 | 17.1 |
| 18.052 | 4.91 | 31.9 |
| 18.483 | 4.796 | 7.8 |
| 18.9 | 4.691 | 8.9 |
| 19.124 | 4.637 | 20.1 |
| 20.303 | 4.37 | 17 |
| 20.626 | 4.303 | 9.5 |
| 21.142 | 4.199 | 10.6 |
| 21.709 | 4.09 | 23 |
| 22.269 | 3.989 | 6.2 |
| 22.854 | 3.888 | 16.2 |
| 23.149 | 3.839 | 9.9 |
| 23.629 | 3.762 | 8.3 |
| 24.92 | 3.57 | 5.2 |
| 25.218 | 3.529 | 6.6 |
| 25.76 | 3.456 | 5 |
| 26.033 | 3.42 | 6.2 |
| 26.325 | 3.383 | 16.1 |
| 28.097 | 3.173 | 7.8 |
| 28.949 | 3.082 | 6.2 |
| 29.974 | 2.9787 | 6.7 |
